(19) United States
(12) Patent Application Publication
(10) Pub. No.: US 2025/0251511 A1
(43) Pub. Date: Aug. 7, 2025

(54) Ultrasound Imaging System Having Near-Infrared/Infrared Detection

(71) Applicant: Bard Access Systems, Inc., Salt Lake City, UT (US)

(72) Inventors: Steffan Sowards, Salt Lake City, UT (US); William Robert McLaughlin, Bountiful, UT (US); Anthony K. Misener, Bountiful, UT (US)

(21) Appl. No.: 19/188,507

(22) Filed: Apr. 24, 2025

Related U.S. Application Data
(63) Continuation of application No. 17/722,111, filed on Apr. 15, 2022, now Pat. No. 12,287,403.
(60) Provisional application No. 63/175,475, filed on Apr. 15, 2021.

(51) Int. Cl. G01S 15/89; G01S 15/86; G01S 17/89; G06T 7/00

(52) U.S. Cl. CPC G01S 15/8906; G01S 15/86; G01S 17/89; G06T 7/0014; G06T 2207/10048; G06T 2207/10132; G06T 2207/30101

(57) ABSTRACT

Disclosed herein is an ultrasound imaging system including an ultrasound probe and a blood vessel visualization device. The ultrasound probe includes an ultrasound generation device and is configured to detect one or more blood vessels. The blood vessel visualization device is configured to project a depiction of the blood vessel topography within a target area. The blood vessel visualization device can include one or more near-infrared/infrared emitters configured to generate infrared/near-infrared waves within the target area, one or more near-infrared/infrared sensors configured to detect the difference in reflective properties of tissue and blood vessels within the target area, and one or more visual light projectors configured to project a blood vessel visualization depiction of the blood vessel topography onto the target area.

### PRIORITY

This application is a continuation of U.S. patent application Ser. No. 17/722,111, filed Apr. 15, 2022, now U.S. Pat. No. 12,287,403, which claims the benefit of priority to U.S. Provisional Application No. 63/175,475, filed Apr. 15, 2021, each of which is incorporated by reference in its entirety into this application.

### BACKGROUND

Readily accessing a blood vessel can require multiple sticks to access the correct blood vessel. Clinicians may use vein visualizers, medical devices that detect subcutaneous blood vessels and project a depiction of the vein topography on the skin surface, to readily access the correct blood vessel. Vein visualizers must be held some distance away from the skin surface where hands or other medical devices may disturb the depiction of the vein topography, adding difficulty to accessing the correct blood vessel. It would be beneficial to clinicians and patients to have a vein visualizer whose depiction would not be disturbed, allowing clinicians ready access to the correct blood vessel. Disclosed herein are an ultrasound imaging system and method of use that address the foregoing.

### SUMMARY

Disclosed herein is an ultrasound imaging system including an ultrasound probe and a blood vessel visualization device. The ultrasound probe includes an ultrasound generation device and is configured to detect one or more blood vessels. The blood vessel visualization device is configured to project a depiction of the blood vessel topography within a target area, the blood vessel visualization device includes one or more near-infrared/infrared emitters configured to generate infrared/near-infrared waves within the target area, one or more near-infrared/infrared sensors configured to detect the difference in reflective properties of tissue and blood vessels within the target area, and one or more visual light projectors configured to project a blood vessel visualization depiction of the blood vessel topography onto the target area.

In some embodiments, the one or more visual light projectors project the blood vessel visualization depiction to one side of the ultrasound probe onto the target area.

In some embodiments, each of the one or more near-infrared sensors, the one or more near-infrared/infrared emitters, and the one or more visual light projectors are coupled to the ultrasound probe.

In some embodiments, the one or more near-infrared/infrared emitters and the one or more near-infrared/infrared sensors are located oblique to an ultrasound acoustic stack of the ultrasound probe.

In some embodiments, the blood vessel visualization device includes a console having non-transitory computer readable medium, an energy source and a plurality of logic modules.

In some embodiments, the console is in communication with each of the ultrasound probe, the one or more near-infrared/infrared emitters, the one or more near-infrared/infrared sensors and the one or more visual light projectors.

In some embodiments, the energy source is in communication with each of the one or more infrared/near-infrared emitters, the one or more infrared/near-infrared sensors, and the one or more visual light projectors.

In some embodiments, the energy source is untethered to one or more of the one or more near-infrared/infrared emitters, the one or more near-infrared/infrared sensors, or the one or more visible light projectors.

In some embodiments, the energy source includes an induction coupling system configured to wirelessly provide energy to the one or more near-infrared/infrared emitters, the one or more near-infrared/infrared sensors or the one or more visible light projectors.

In some embodiments, the energy source is tethered to one or more of the infrared/near-infrared emitters, the infrared/near-infrared sensors or the visible light projectors.

In some embodiments, the plurality of logic modules, when executed by the processor, are configured to perform operations including receiving ultrasound data from the ultrasound probe, correlating a detected location of one or more blood vessels with a starting location for a blood vessel visualization depiction within the target area, activating each of the near-infrared/infrared emitters, the near-infrared/infrared sensors, and the visual light projectors, receiving detected near-infrared/infrared data from the near-infrared/infrared sensors, and generating and projecting to one side of the ultrasound probe, the blood vessel visualization depiction within the target area.

In some embodiments, the ultrasound generation device includes a microelectromechanical systems based device.

In some embodiments, the ultrasound probe includes the ultrasound generation device, the one or more near-infrared/infrared sensors and the one or more visual light projectors, and a vascular access device includes the one or more near-infrared/infrared emitters, the vascular access device configured to be inserted into one of the detected blood vessels within the target area.

In some embodiments, the vascular access device includes a visual indicator configured to be activated based upon a vascular access device trajectory relative to a target blood vessel or an identified blood vessel type including an artery or a vein.

Also disclosed herein is a method of detecting one or more blood vessels in a target area and generating and projecting a blood vessel visualization depiction over the target area. The method includes detecting, by ultrasound and near-infrared/infrared electromagnetic waves, blood vessel topography in the target area, generating a blood vessel visualization depiction, and projecting, to one side of an ultrasound probe, the blood vessel visualization depiction on the target area.

In some embodiments, detecting by near-infrared/infrared electromagnetic waves includes one or more near-infrared/infrared sensors detecting a difference in reflective properties of tissue and blood vessels within the target area.

In some embodiments, detecting by near-infrared/infrared electromagnetic waves includes the electromagnetic waves being emitted from one or more near-infrared/infrared emitters coupled to the ultrasound probe and being detected by the one or more near-infrared/infrared sensors coupled to the ultrasound probe.

In some embodiments, detecting by near-infrared/infrared electromagnetic waves includes the electromagnetic waves being emitted from one or more near-infrared/infrared emitters coupled to a vascular access device and being detected by one or more near-infrared/infrared sensors coupled to the ultrasound probe.

In some embodiments, generating a blood vessel visualization depiction includes a console in communication with each of the ultrasound probe, the one or more near-infrared/infrared emitters and the near-infrared/infrared sensors generating the blood vessel visualization depiction using ultrasound data received from the ultrasound probe and electromagnetic wave data received from the one or more near-infrared/infrared sensors.

In some embodiments, projecting, to one side of an ultrasound probe, the blood vessel visualization depiction on the target area includes one or more visual light projectors coupled to the ultrasound probe projecting the blood vessel visualization depiction onto the target area.

These and other features of the concepts provided herein will become more apparent to those of skill in the art in view of the accompanying drawings and following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a cross sectional view of an ultrasound imaging system including an ultrasound probe having a blood vessel visualization device coupled thereto, in accordance with some embodiments.

FIG. 2 illustrates a block diagram of some components of the ultrasound imaging system including the ultrasound probe having a console, in accordance with some embodiments.

FIG. 3 illustrates a cross sectional view of the ultrasound imaging system including the ultrasound probe and a vascular access device, in accordance with some embodiments.

FIGS. 4A-4C illustrate a side cross-sectional view of an exemplary method of generating and projecting a blood vessel visualization depiction over a target area, in accordance with some embodiments.

FIG. 5 illustrates a flow chart of an exemplary method of detecting one or more blood vessels in a target are, generating and projecting a blood vessel visualization depiction over the target area, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Unless defined otherwise, all technical and scientific terms used herein have the same meaning as commonly understood by those of ordinary skill in the art.

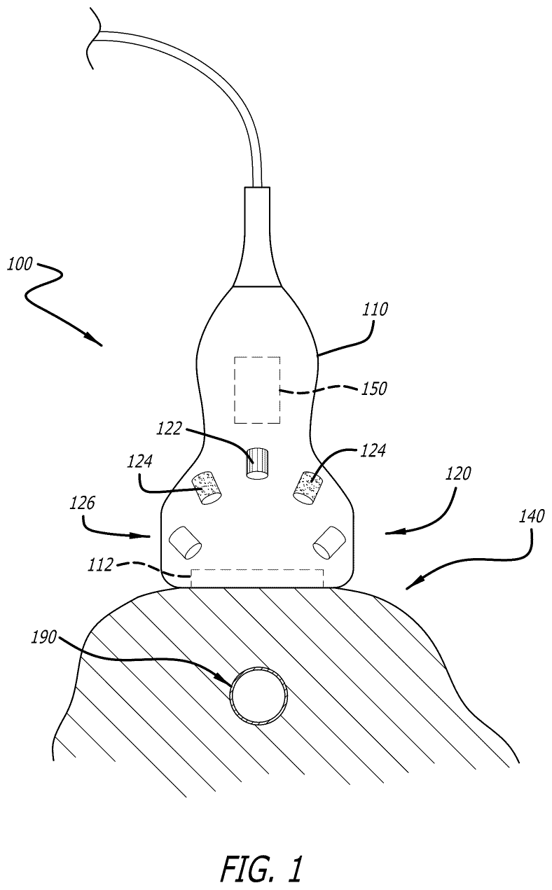

FIG. 1 illustrates a perspective view of an ultrasound imaging system (“system”) 100 including an ultrasound probe 110 having a blood vessel visualization device 120 coupled thereto, in accordance with some embodiments. In some embodiments, the ultrasound probe 110 may be configured to detect one or more blood vessels within a target area 140 by contacting a skin surface within the target area 140. In some embodiments, the ultrasound probe 110 may include an ultrasound generation device 112 including an ultrasound acoustic stack or other various modalities of ultrasound generation (e.g., microelectromechanical systems (MEMS) based, etc.). In some embodiments, the target area 140 may include the one or more blood vessels 190 to be accessed by a vascular access device. The blood vessel visualization device 120 may be configured to sense subcutaneous blood vessel topography and project, to one side of the ultrasound probe 110, a depiction of the sensed blood vessel topography on the skin surface of the target area 140, allowing a clinician to access a blood vessel without disturbing the depiction.

In some embodiments, the blood vessel visualization device 120 includes one or more near-infrared/infrared (“NIR/IR”) sensors 122, one or more NIR/IR emitters 124, and one or more visual light projectors 126. The NIR/IR sensors 122, the NIR/IR emitters 124 and the visual light projectors 126 may all be in communication with a console 150. In some embodiments, the NIR/IR sensors 122, the NIR/IR emitters 124 and the visual light projectors 126 may be in wireless communication with the console 150. In some embodiments, the console 150 may be in communication with the ultrasound probe 110 including in communication with the ultrasound generation device 112. In some embodiments, the console 150 may be coupled to the ultrasound probe 110 or integrated into the ultrasound probe 110. In some embodiments, the NIR/IR emitters 124 may be configured to emit electromagnetic waves within the near-infrared/infrared spectrum within the target area 140. The electromagnetic waves reflect off of subcutaneous structures such as blood vessels. In some embodiments, the NIR/IR sensors 122 may be configured to detect the reflected NIR/IR electromagnetic waves and transmit the detected NIR/IR data to the console 150. In some embodiments, the NIR/IR sensors 122 may be configured to detect the difference in reflective properties of tissue and blood vessels within the target area 140. The console 150 may be configured to receive the detected NIR/IR data, correlate the difference in electromagnetic reflectance between blood vessels and surrounding tissues to generate a blood vessel visualization depiction. The one or more visual light projectors 126 may be configured to project the blood vessel visualization depiction to one side of the ultrasound probe 110 on the skin surface within the target area 140. In some embodiments, the NIR/IR emitters 124 and the NIR/IR sensors 122 may be located oblique to the ultrasound acoustic stack 112.

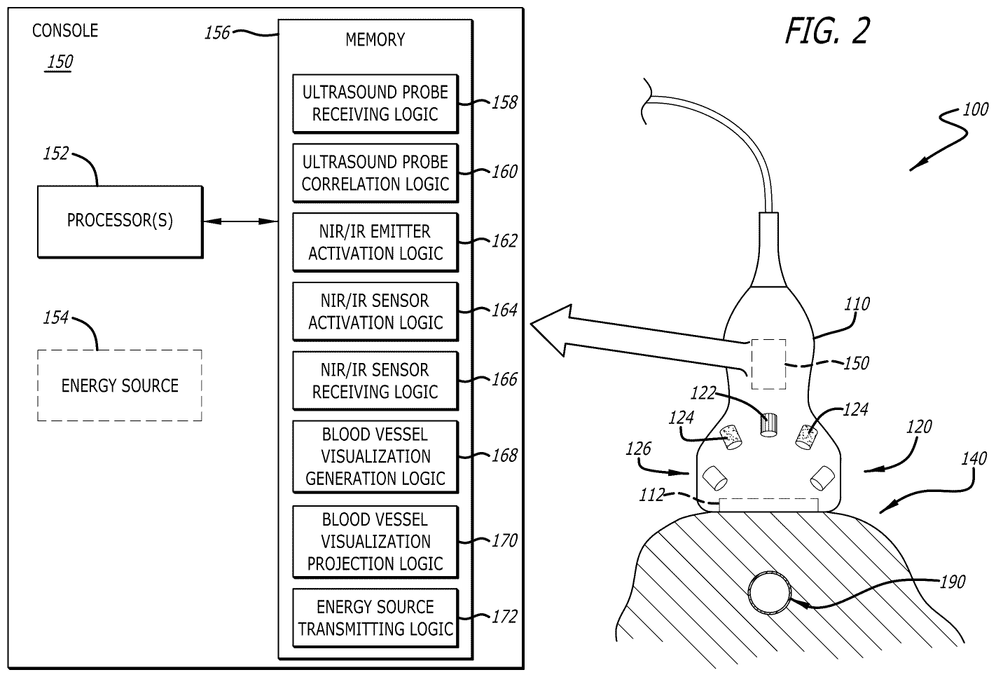

FIG. 2 illustrates a block diagram of some components of the ultrasound imaging system 100 including the console 150, in accordance with some embodiments. In some embodiments, the console 150 may be coupled to or integrated into the ultrasound probe 110. In some embodiments, the console 150 may be in communication with the ultrasound probe 110 and be configured to receive the ultrasound data to generate a portion of the blood vessel visualization depiction within the target area 140. The blood vessel visualization device 120 may include the console 150 in communication with each of: the one or more blood vessel sensors 122, the one or more blood vessel emitters 124, the visible light projectors 126 and the ultrasound probe 110. In some embodiments, the console 150 may be in wireless communication with each of the blood vessel sensors 122, the blood vessel emitters 124, the visible light projectors 126 and the ultrasound probe 110. Exemplary wireless communication modalities can include WiFi, Bluetooth, Near Field Communications (NFC), cellular Global System for Mobile Communication (“GSM”), electromagnetic (EM), radio frequency (RF), combinations thereof, or the like.

In some embodiments, the console 150 may include one or more processors 152, an energy source 154, non-transitory computer readable medium (“memory”) 156 and a plurality of logic modules. The plurality of logic modules may include one or more of: a ultrasound probe receiving logic 158, a ultrasound probe correlation logic 160, a NIR/IR sensor activation logic 162, a NIR/IR emitter activation logic 164, a NIR/IR sensor receiving logic 166, a blood vessel visualization generation logic 168, a blood vessel visualization projection logic 170 and an energy source transmitting logic 172. In some embodiments, the ultrasound probe receiving logic 158 may be configured to receive the ultrasound wave data from the ultrasound probe 110 including from the ultrasound generation device 112. In some embodiments, the ultrasound probe correlation logic 160 may be configured to correlate the detected location of the one or more blood vessels 190 with a starting location for the blood vessel visualization depiction within the target area 140. In some embodiments, the NIR/IR emitter activation logic 164 may be configured to activate the NIR/IR emitters 124 to emit electromagnetic waves within the target area 140. In some embodiments, the NIR/IR sensor activation logic 162 may be configured to activate the NIR/IR sensors 122. In some embodiments, the NIR/IR sensor receiving logic 166 may be configured to detect the reflected electromagnetic waves within the target area 140. In some embodiments, the NIR/IR sensor receiving logic 166 may be configured to detect the difference in the reflective properties of tissue and blood vessels 190 within the target area 140. In some embodiments, the blood vessel visualization generation logic 168 may be configured to generate the blood vessel visualization depiction. The blood vessel visualization generation logic 168 may be configured to receive the detected electromagnetic wave data and the detected ultrasound wave data to generate the blood vessel visualization depiction of the blood vessels 190 within the target area 140. In some embodiments, the blood vessel visualization depiction of the blood vessels 190 within the target area 140 may include a topographical map depicting the blood vessels 190 and surrounding tissues. In some embodiments, the blood vessel visualization projection logic 170 may be configured to use the one or more visible light projectors 126 to project the blood vessel visualization depiction on the target area 140.

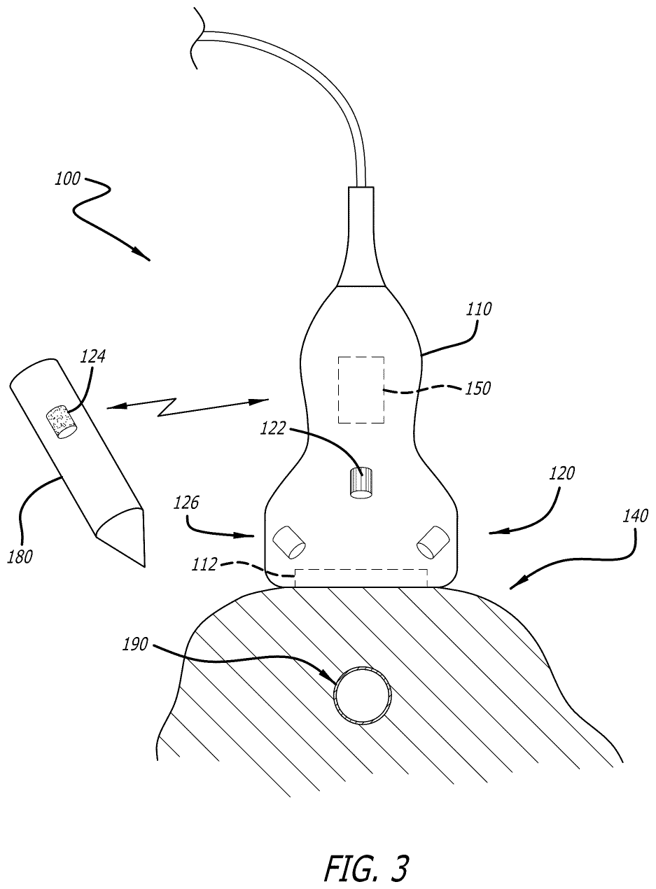

In some embodiments, the energy source 154 may be configured to provide power to the one or more NIR/IR emitters 124, the one or more NIR/IR sensors 122 and the one or more visible light projectors 126. In some embodiments, the NIR/IR emitters 124, the NIR/IR sensors 122, or the visible light projectors 126 may be tethered to the energy source 154. In some embodiments, the energy source 154 may be in communication with one or more of the NIR/IR sensors 122, the NIR/IR emitters 124 and the visible light projectors 126. In some embodiments, the energy source 154 may include a battery coupled to one or more of the NIR/IR emitters 124, the NIR/IR sensors 122 and the visible light projectors 126. In some embodiments, the NIR/IR sensors 122 or the NIR/IR emitters 124 may be untethered to the ultrasound probe 110 [e.g., coupled to a vascular access device 180 (see FIG. 3)]. The energy source 154 may then be configured to wirelessly provide power to either the NIR/IR sensors 122 or the NIR/IR emitters 124. The energy source 154 may be configured to wirelessly provide power to the NIR/IR sensors 122 through an induction coupling system. In this embodiment, the plurality of logic modules may be configured to include an energy source transmitting logic 172 configured to regulate the amount of power that is wirelessly provided to the NIR/IR sensors 122 or NIR/IR emitters 124.

FIG. 3 illustrates a cross sectional view of the ultrasound imaging system 100 wherein the NIR/IR emitters 124 are coupled to a vascular access device 180 and the NIR/IR sensors 122 and visual light projectors 126 are coupled to the ultrasound probe 110. As used herein a “vascular access device” can include a catheter, peripherally inserted central catheter (“PICC”), peripheral intravenous line (“PIV”), central venous catheter (“CVC”), midline catheter, a needle, or the like. In some embodiments, the NIR/IR emitters 124 and the NIR/IR sensors 122 may be separated between the ultrasound probe 110 and the vascular access device 180. In some embodiments, the NIR/IR emitters 124 may be coupled to the vascular access device 180 and the NIR/IR sensors 122 may be coupled to the ultrasound probe 110. In some embodiments, the NIR/IR emitters 124 may be coupled to the ultrasound probe 110 and the NIR/IR sensors 122 may be coupled to the vascular access device 180. In some embodiments, the NIR/IR emitters 124 may be integrated into the vascular access device 180. In this embodiment, wherein either the NIR/IR emitters 124 or the NIR/IR sensors 122 are coupled to the vascular access device 180, the NIR/IR emitters or NIR/IR sensors 122 may be powered through induction coupling. In an embodiment, the vascular access device 180 is enabled with a visual indicator, wherein the visual indicator is activated based upon the trajectory of the vascular access device 180 relative to a target blood vessel or the visual indicator is activated based upon the identified blood vessel type. The console 150 functions as described above.

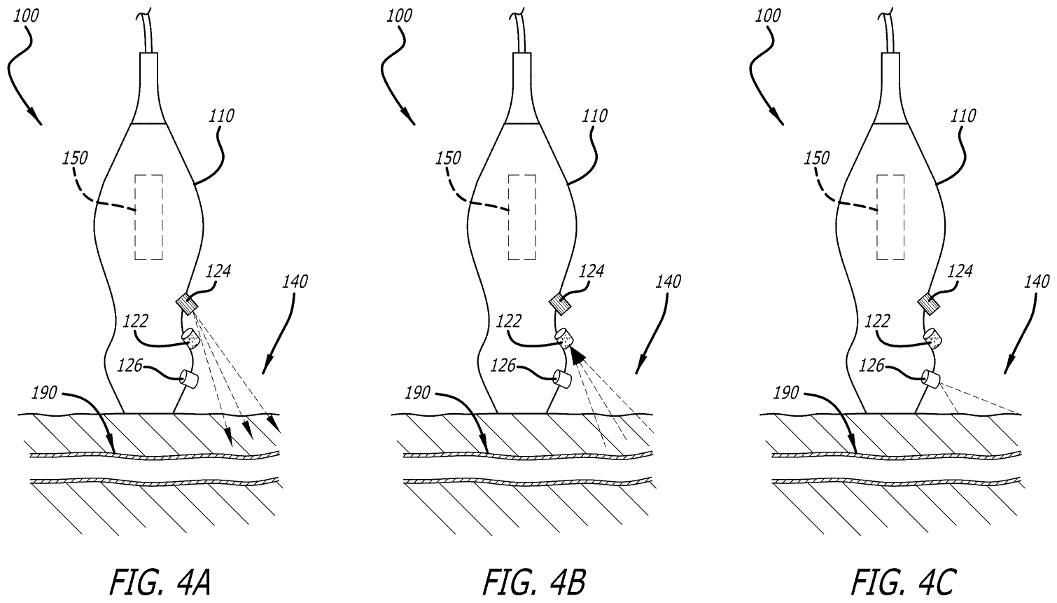

FIGS. 4A-4C illustrates a side cross-sectional view of an exemplary method of detecting blood vessel topography and projecting a blood vessel visualization depiction over the target area 140, in accordance with some embodiments. The ultrasound probe 110 includes the NIR/IR emitters 124, the NIR/IR sensors 122 and the visual light projectors 126, as described above. As illustrated in FIG. 4A, in some embodiments, the ultrasound imaging system 100 including the ultrasound probe 110 may be configured to detect one or more blood vessels 190 within the target area 140. In some embodiments, the ultrasound probe 110 may be configured to detect the one or more blood vessels 190 within target area 140 by ultrasound. The NIR/IR emitters 124 may emit electromagnetic waves subcutaneously within the target area 140. As illustrated in FIG. 4A, in some embodiments, the NIR/IR emitters 124, the NIR/IR sensors 122, and the visual light projectors 126 may be arranged in various configurations on the ultrasound probe 110. In some embodiments, the NIR/IR emitters 124 may be located above the NIR/IR sensors 122, and the NIR/IR sensors 122 may be located above the visual light projectors 126. In some embodiments, the NIR/IR emitters 124 may be located adjacent to the NIR/IR sensors 122. In some embodiments, the visual light projectors 126 may be located adjacent to the NIR/IR sensors 122 or the NIR/IR emitters 124.

As illustrated in FIG. 4B, the NIR/IR sensors 122 may be configured to detect the reflected electromagnetic waves, including the difference in reflective properties of the tissue and blood vessels within the target area 140. The NIR/IR sensors 122 may transmit the detected electromagnetic waves to the console 150 where the console 150 may use the detected electromagnetic wave data, along with the detected blood vessel data within the target area 140 to generate a blood vessel visualization depiction. As illustrated in FIG. 4C, the visual light projectors 126 may be configured to receive the blood vessel visualization depiction and project the blood vessel visualization depiction onto the target area 140 including onto a skin surface. A user may use the blood vessel visualization depiction to accurately access the blood vessel 190 within the target area 140.

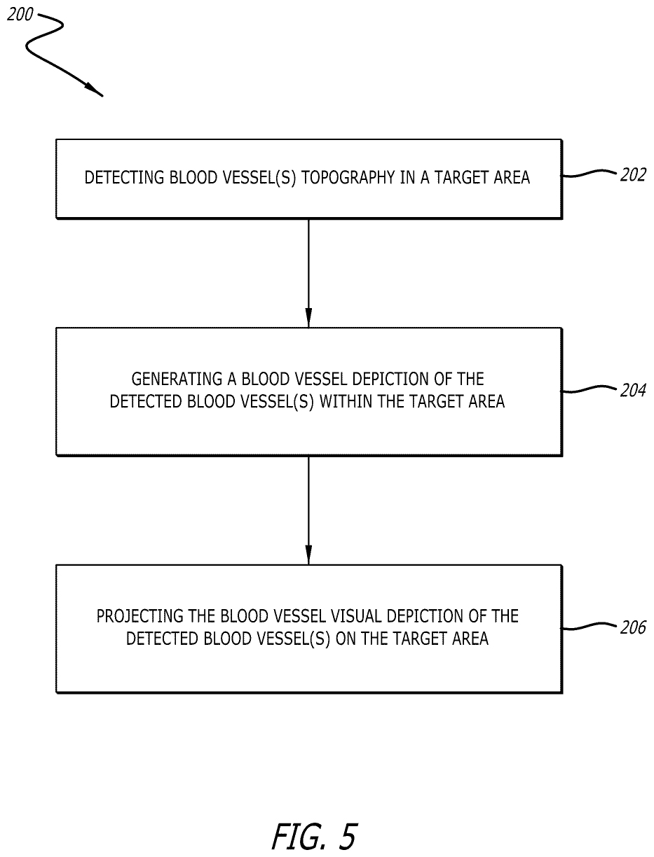

FIG. 5 illustrates a flow chart of the method 200 of detecting one or more blood vessels 190 in a target area 140 and generating and projecting a blood vessel visualization depiction over the target area 140, in accordance with some embodiments. In some embodiments, the method 200 includes detecting blood vessel topography in a target area 140 (block 202). In some embodiments, detecting blood vessel topography in the target area 140 includes detecting, by near-infrared/infrared electromagnetic waves, blood vessel topography in the target area 140. In some embodiments, detecting blood vessel topography in the target area 140 includes detecting, by ultrasound, blood vessel topography in the target area 140. In some embodiments, detecting blood vessel topography in the target area 140 includes detecting by ultrasound and near-infrared/infrared electromagnetic waves, blood vessel topography in the target area 140. In some embodiments, detecting blood vessel topography includes using the NIR/IR emitters 124 configured to emit near-infrared/infrared electromagnetic waves and NIR/IR sensors 122 coupled to the ultrasound probe 110 configured to detect the near-infrared/infrared electromagnetic waves to detect the blood vessel topography. In some embodiments, detecting blood vessel topography includes using the NIR/IR emitters 124 couped to the vascular access device 180 configured to emit near-infrared/infrared electromagnetic waves and the NIR/IR sensors 122 coupled to the ultrasound probe 110 configured to detect the near-infrared/infrared electromagnetic waves to detect the blood vessel topography. In some embodiments, detecting blood vessel topography in the target area 140 includes the NIR/IR sensors 122 detecting the difference in reflective properties of tissue and blood vessels within the target area 140. The method 200 further includes generating a blood vessel visual depiction of the detected blood vessels 190 within the target area 140 (block 204). In some embodiments, generating the blood vessel visual depiction includes the console 150 generating the blood vessel visual depiction. In some embodiments, the console 150 may use blood vessel topography data received from the NIR/IR sensors 122 to generate the blood vessel visual depiction. In some embodiments, the console 150 may also use ultrasound data received from the ultrasound probe 110 to generate the blood vessel visual depiction. In some embodiments, the method 200 includes projecting, to one side of the ultrasound probe 110, the blood vessel visual depiction of the detected blood vessel on the target area 140 (block 206). In some embodiments projecting the blood vessel visual depiction of the detected blood vessels on the target area 140 includes the blood vessel visual depiction having distinct colors indicating different blood vessel type (e.g., artery versus vein).

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An ultrasound imaging system, comprising:
an ultrasound probe configured to detect one or more blood vessels, the ultrasound probe including an ultrasound generation device operable through a skin contacting side of the ultrasound probe; and
a blood vessel visualization device configured to project a depiction of blood vessel topography within a target area, the blood vessel visualization device coupled to a side of the ultrasound probe adjacent to the skin contacting side, the blood visualization device comprising:
one or more near-infrared/infrared emitters configured to generate infrared/near-infrared waves in the target area;
one or more near-infrared/infrared sensors configured to detect a difference in reflective properties of tissue and blood vessels in the target area; and
one or more visual light projectors configured to project a blood vessel visualization depiction of the blood vessel topography onto the target area.

2. The ultrasound imaging system according to claim 1, wherein the one or more visual light projectors project the blood vessel visualization depiction to one side of the ultrasound probe onto the target area.

3. The ultrasound imaging system according to claim 1, wherein the one or more near-infrared/infrared emitters and the one or more near-infrared/infrared sensors are located oblique to an ultrasound acoustic stack of the ultrasound probe.

4. The ultrasound imaging system according to claim 1, wherein the blood vessel visualization device includes a console having non-transitory computer readable medium, an energy source and a plurality of logic modules.

5. The ultrasound imaging system according to claim 4, wherein the console is in communication with each of the ultrasound probe, the one or more near-infrared/infrared emitters, the one or more near-infrared/infrared sensors and the one or more visual light projectors.

6. The ultrasound imaging system according to claim 5, wherein the energy source is in communication with each of the one or more near-infrared/infrared emitters, the one or more near-infrared/infrared sensors, and the one or more visual light projectors.

7. The ultrasound imaging system according to claim 6, wherein the energy source is untethered to one or more of the one or more near-infrared/infrared emitters, the one or more near-infrared/infrared sensors, or the one or more visible light projectors.

8. The ultrasound imaging system according to claim 6, wherein the energy source includes an induction coupling system configured to wirelessly provide energy to the one or more near-infrared/infrared emitters, the one or more near-infrared/infrared sensors or the one or more visible light projectors.

9. The ultrasound imaging system according to claim 6, wherein the energy source is tethered to the one or more near-infrared/infrared emitters, the one or more near-infrared/infrared sensors or the one or more visible light projectors.

10. The ultrasound imaging system according to claim 4, wherein the plurality of logic modules, when executed by one or more processors, are configured to perform operations:
receiving ultrasound data from the ultrasound probe;
correlating a detected location of the one or more blood vessels with a starting location for the blood vessel visualization depiction within the target area;
activating each of the one or more near-infrared/infrared emitters, the one or more near-infrared/infrared sensors, and the one or more visual light projectors;
receiving detected near-infrared/infrared data from the one or more near-infrared/infrared sensors; and
generating and projecting to one side of the ultrasound probe, the blood vessel visualization depiction within the target area.

11. The ultrasound imaging system according to claim 1, wherein the ultrasound generation device includes a microelectromechanical systems based device.

12. The ultrasound imaging system according to claim 1, further comprising a vascular access device including a visual indicator configured to be activated based upon a vascular access device trajectory relative to a target blood vessel or an identified blood vessel type including an artery or a vein.
